(19) United States
(12) Patent Application Publication
(10) Pub. No.: US 2015/0018917 A1
(43) Pub. Date: Jan. 15, 2015

(54) CONDUCTOR ARRANGEMENTS FOR ELECTRICAL STIMULATION LEADS AND SYSTEMS AND METHODS UTILIZING THE LEADS

(71) Applicant: BOSTON SCIENTIFIC NEUROMODULATION CORPORATION, Valencia, CA (US)

(72) Inventors: David Ernest Wechter, Santa Clarita, CA (US); Joshua Dale Howard, Chatsworth, CA (US); Michael X. Govea, Glendale, CA (US); Anne Margaret Pianca, Santa Monica, CA (US)

(21) Appl. No.: 14/326,267

(22) Filed: Jul. 8, 2014

Related U.S. Application Data
(60) Provisional application No. 61/846,470, filed on Jul. 15, 2013.

(51) Int. Cl. A61N 1/08; A61N 1/05

(52) U.S. Cl. CPC A61N 1/08; A61N 1/05; A61N 2001/086
USPC 607/116

(57) ABSTRACT

An electrical stimulation lead includes at least one lead body; electrodes disposed along the distal end portion of the at least one lead body; terminals disposed along the proximal end portion of the at least one lead body; and conductors electrically coupling the terminals to the electrodes. Each of the conductors forms at least one current suppression unit. Each current suppression unit includes a first conductor segment wound in a coil extending in a forward direction followed by a second conductor segment wound in a coil extending in a reverse direction followed by a third conductor segment wound in a coil extending in the forward direction. The first and third conductor segments are wound with a first coil diameter and the second conductor segment is also wound with the first coil diameter except at positions where the second conductor segment overlaps either the first or third conductor segments.

### CROSS-REFERENCE TO RELATED APPLICATIONS

This application claims the benefit under 35 U.S.C. §119(e) of U.S. Provisional Patent Application Ser. No. 61/846,470, filed Jul. 15, 2013, which is incorporated herein by reference.

### FIELD

The present invention is directed to the area of implantable electrical stimulation systems and methods of making and using the systems. The present invention is also directed implantable electrical stimulation leads having conductor arrangements that resist induction of current by external electromagnetic fields, as well as methods of making and using the leads and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is an electrical stimulation lead including at least one lead body having a distal end portion, a proximal end portion, and a longitudinal length; electrodes disposed along the distal end portion of the at least one lead body; terminals disposed along the proximal end portion of the at least one lead body; and conductors electrically coupling the terminals to the electrodes. Each of the conductors forms at least one current suppression unit. Each current suppression unit includes a first conductor segment wound in a coil and extending in a forward direction from a first point to a second point, a second conductor segment wound in a coil and extending in a reverse direction from the second point to a third point, and a third conductor segment wound in a coil and extending in the forward direction from the third point to a fourth point. The first and third conductor segments are wound around a longitudinal axis with a first coil diameter and the second conductor segment is also wound around the longitudinal axis with the first coil diameter except at positions where the second conductor segment overlaps either the first conductor segment or the third conductor segment.

Another embodiment is an electrical stimulation lead including at least one lead body having a distal end portion, a proximal end portion, and a longitudinal length; electrodes disposed along the distal end portion of the at least one lead body; terminals disposed along the proximal end portion of the at least one lead body; and conductors electrically coupling the terminals to the electrodes. Each of the conductors forms at least one current suppression unit. Each current suppression unit includes a first conductor segment wound in a coil in a first winding direction selected from clockwise or counterclockwise, a transition segment connected to the first conductor segment, and a second conductor segment wound in a coil in a second winding direction that is opposite the first winding direction.

Yet another embodiment is an electrical stimulation lead including at least one lead body having a distal end portion, a proximal end portion, and a longitudinal length; electrodes disposed along the distal end portion of the at least one lead body; terminals disposed along the proximal end portion of the at least one lead body; and conductors electrically coupling the plurality of terminals to the plurality of electrodes. The conductors include a first group containing two or more of the conductors and a second group containing two or more of the conductors. The first group of conductors are co-wound in a coil in a first winding direction to form a first layer and the second group of conductor are co-wound in a coil in a second winding direction to form a second layer disposed over the first layer. The first winding direction is selected from clockwise or counterclockwise and the second winding direction is opposite the first winding direction.

A further embodiment is an electrical stimulation system that includes any of the electrical stimulation leads described above and a control unit coupleable to the electrical stimulation lead.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of an electrical stimulation system that includes a paddle lead electrically coupled to a control module, according to the invention;

FIG. 2 is a schematic side view of one embodiment of an electrical stimulation system that includes a percutaneous lead electrically coupled to a control module, according to the invention;

FIG. 3A is a schematic side view of one embodiment of the control module of FIG. 1 configured and arranged to electrically couple to an elongated device, according to the invention;

FIG. 3B is a schematic side view of one embodiment of a lead extension configured and arranged to electrically couple the elongated device of FIG. 2 to the control module of FIG. 1, according to the invention;

FIG. 4 is a schematic side view of a previous arrangement of conductors of the lead formed in current suppression units;

FIG. 5A is a schematic side view of one embodiment of a new arrangement of conductors of the lead formed in current suppression units, according to the invention;

FIG. 5B is a schematic cross-sectional view of the arrangement of conductors of FIG. 5A at a position along the lead where conductor segments do not overlap, according to the invention;

FIG. 5C is a schematic cross-sectional view of the arrangement of conductors of FIG. 5A at a position along the lead where conductor segments do overlap, according to the invention;

FIG. 6 is a schematic side view of a second embodiment of a new arrangement of conductors of the lead formed in current suppression units, according to the invention;

FIG. 7 is a schematic side view of one embodiment of an arrangement of conductors of the lead formed in current suppression units with two layers of conductor wound in opposite directions, according to the invention;

FIG. 8 is a schematic side view of one embodiment of an arrangement of conductors of the lead formed in sections with opposite directions of winding, according to the invention;

FIG. 9 is a schematic side view of one embodiment of a conductor of the lead formed in sections with opposite directions of winding, according to the invention;

FIG. 10 is a schematic cross-sectional view of one embodiment of a multi-lumen conductor guide of a lead that can receive a conductor such as that illustrated in FIG. 9 (but without the jacket) within each conductor lumen, according to the invention;

FIG. 11A is a schematic side view of one embodiment of a conductor of the lead formed in sections with opposite directions of winding and disposed within a non-conductive tube, according to the invention;

FIG. 11B is a schematic cross-sectional view of one embodiment of a portion of a lead that contains multiple conductor/tube arrangements, such as that illustrated in FIG. 11A, arranged concentrically, according to the invention; and

FIG. 12 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed implantable electrical stimulation leads having conductor arrangements that resist induction of current by external electromagnetic fields, as well as methods of making and using the leads and electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; and 8,391,985; U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615, all of which are incorporated by reference.

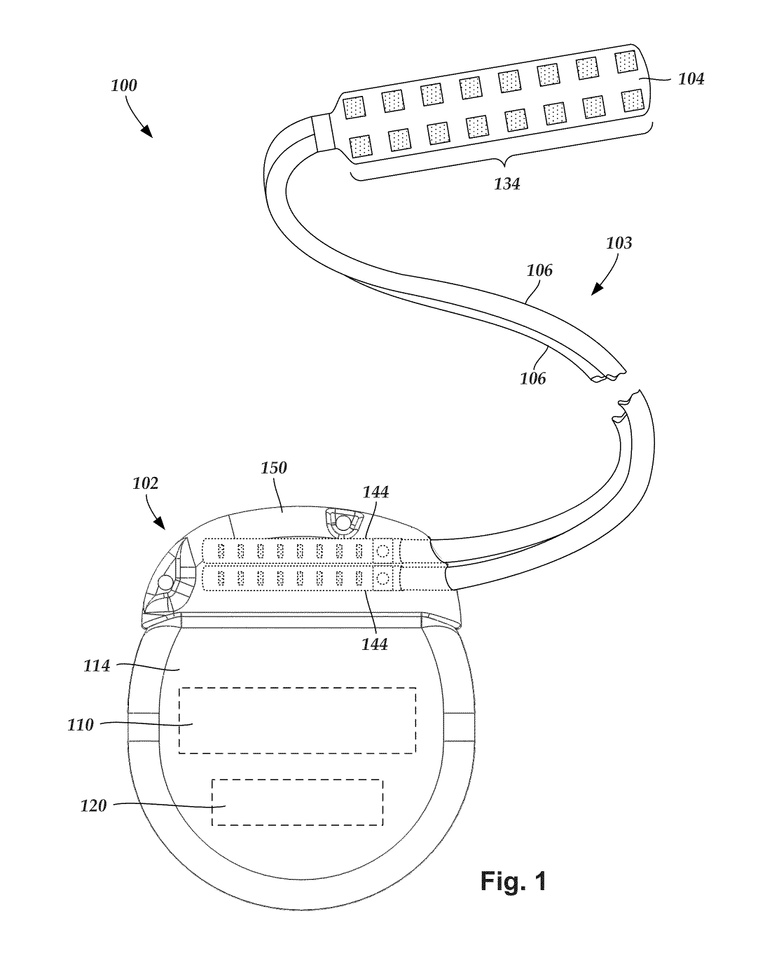

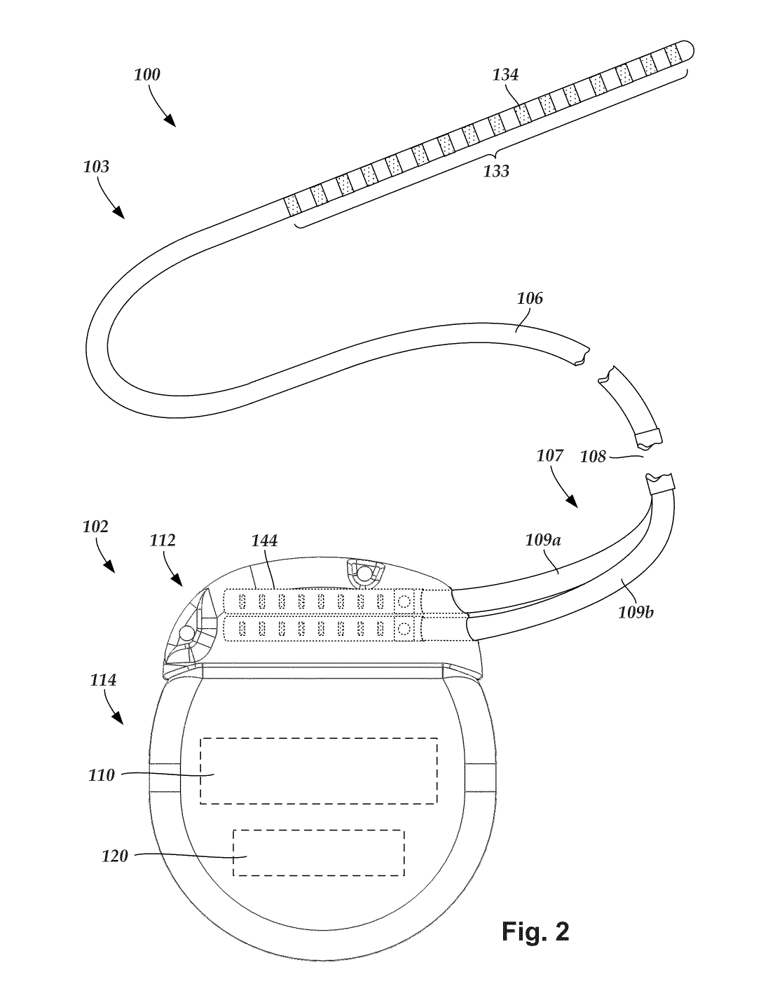

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a paddle body 104 and one or more lead bodies 106. In FIG. 1, the lead 103 is shown having two lead bodies 106. It will be understood that the lead 103 can include any suitable number of lead bodies including, for example, one, two, three, four, five, six, seven, eight or more lead bodies 106. An array of electrodes 133, such as electrode 134, is disposed on the paddle body 104, and an array of terminals (e.g., 210 in FIG. 2A-2B) is disposed along each of the one or more lead bodies 106.

It will be understood that the electrical stimulation system can include more, fewer, or different components and can have a variety of different configurations including those configurations disclosed in the electrical stimulation system references cited herein. For example, instead of a paddle body, the electrodes can be disposed in an array at or near the distal end of a lead body forming a percutaneous lead.

FIG. 2 illustrates schematically another embodiment of the electrical stimulation system 100, where the lead 103 is a percutaneous lead. In FIG. 2, the electrodes 134 are shown disposed along the one or more lead bodies 106. In at least some embodiments, the lead 103 is isodiametric along a longitudinal length of the lead body 106.

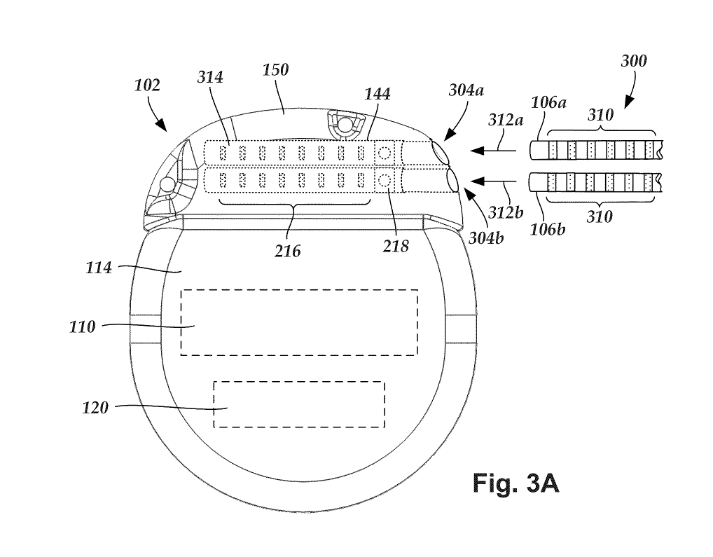

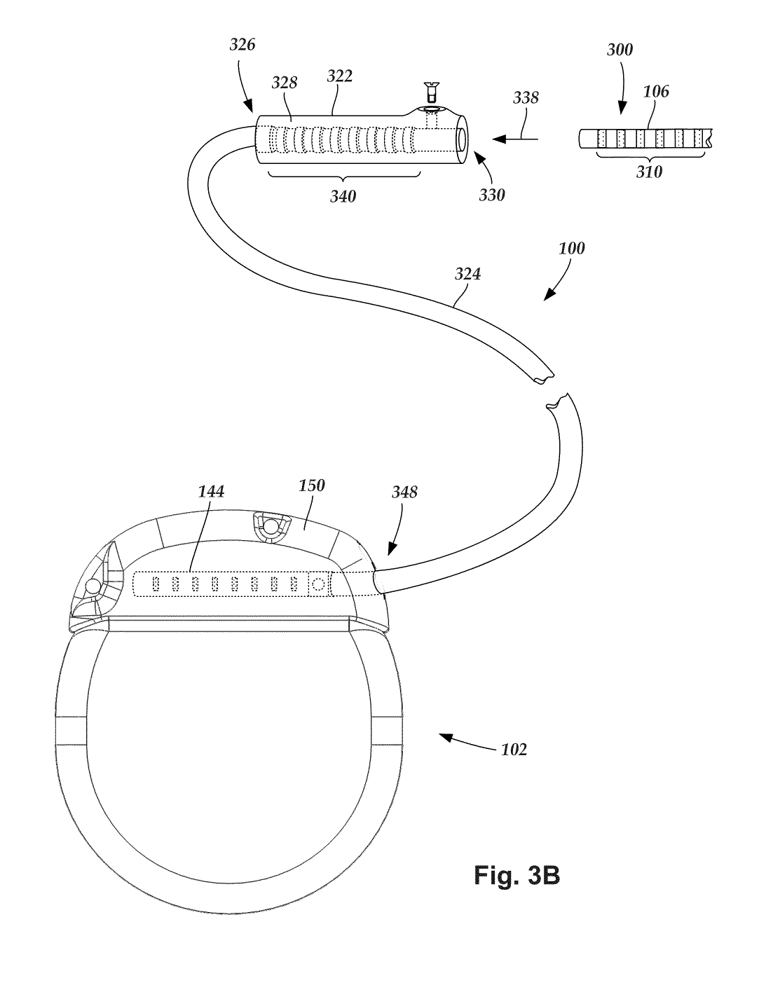

The lead 103 can be coupled to the control module 102 in any suitable manner. In FIG. 1, the lead 103 is shown coupling directly to the control module 102. In at least some other embodiments, the lead 103 couples to the control module 102 via one or more intermediate devices (300 in FIGS. 3A-3B). For example, in at least some embodiments one or more lead extensions 324 (see e.g., FIG. 3B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 2, the electrical stimulation system 100 is shown having a splitter 207 configured and arranged for facilitating coupling of the lead 103 to the control module 102. The splitter 207 includes a splitter connector 208 configured to couple to a proximal end of the lead 103, and one or more splitter tails 209a and 209b configured and arranged to couple to the control module 102 (or another splitter, a lead extension, an adaptor, or the like).

The control module 102 typically includes a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

The electrical stimulation system or components of the electrical stimulation system, including the paddle body 104, the one or more of the lead bodies 106, and the control module 102, are typically implanted into the body of a patient. The electrical stimulation system can be used for a variety of applications including, but not limited to deep brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

Any suitable number of electrodes 134 can be disposed on the lead including, for example, four, five, six, seven, eight, nine, ten, eleven, twelve, fourteen, sixteen, twenty-four, thirty-two, or more electrodes 134. In the case of paddle leads, the electrodes 134 can be disposed on the paddle body 104 in any suitable arrangement. In FIG. 1, the electrodes 134 are arranged into two columns, where each column has eight electrodes 134.

The electrodes of the paddle body 104 (or one or more lead bodies 106) are typically disposed in, or separated by, a non-conductive, biocompatible material such as, for example, silicone, polyurethane, polyetheretherketone (“PEEK”), epoxy, and the like or combinations thereof. The one or more lead bodies 106 and, if applicable, the paddle body 104 may be formed in the desired shape by any process including, for example, molding (including injection molding), casting, and the like. The non-conductive material typically extends from the distal ends of the one or more lead bodies 106 to the proximal end of each of the one or more lead bodies 106.

In the case of paddle leads, the non-conductive material typically extends from the paddle body 104 to the proximal end of each of the one or more lead bodies 106. Additionally, the non-conductive, biocompatible material of the paddle body 104 and the one or more lead bodies 106 may be the same or different. Moreover, the paddle body 104 and the one or more lead bodies 106 may be a unitary structure or can be formed as two separate structures that are permanently or detachably coupled together.

Terminals (e.g., 310 in FIGS. 3A-3B) are typically disposed along the proximal end of the one or more lead bodies 106 of the electrical stimulation system 100 (as well as any splitters, lead extensions, adaptors, or the like) for electrical connection to corresponding connector contacts (e.g., 314 in FIGS. 3A-3B). The connector contacts are disposed in connectors (e.g., 144 in FIGS. 1-3B; and 322 FIG. 3B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 3A is a schematic side view of one embodiment of a proximal end of one or more elongated devices 300 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 3B, an adaptor, or the like or combinations thereof), or a combination thereof.

The control module connector 144 defines at least one port into which a proximal end of the elongated device 300 can be inserted, as shown by directional arrows 312a and 312b. In FIG. 3A (and in other figures), the connector housing 112 is shown having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated device 300 is inserted into the ports 304a and 304b, the connector contacts 314 can be aligned with a plurality of terminals 310 disposed along the proximal end(s) of the elongated device(s) 300 to electrically couple the control module 102 to the electrodes (134 of FIG. 1) disposed on the paddle body 104 of the lead 103. Examples of connectors in control modules are found in, for example, U.S. Pat. Nos. 7,244,150 and 8,224,450, which are incorporated by reference.

FIG. 3B is a schematic side view of another embodiment of the electrical stimulation system 100. The electrical stimulation system 100 includes a lead extension 324 that is configured and arranged to couple one or more elongated devices 300 (e.g., one of the lead bodies 106 of FIGS. 1 and 2, the splitter 207 of FIG. 2, an adaptor, another lead extension, or the like or combinations thereof) to the control module 102. In FIG. 3B, the lead extension 324 is shown coupled to a single port 304 defined in the control module connector 144. Additionally, the lead extension 324 is shown configured and arranged to couple to a single elongated device 300. In alternate embodiments, the lead extension 324 is configured and arranged to couple to multiple ports 304 defined in the control module connector 144, or to receive multiple elongated devices 300, or both.

A lead extension connector 322 is disposed on the lead extension 324. In FIG. 3B, the lead extension connector 322 is shown disposed at a distal end 326 of the lead extension 324. The lead extension connector 322 includes a connector housing 328. The connector housing 328 defines at least one port 330 into which terminals 310 of the elongated device 300 can be inserted, as shown by directional arrow 338. The connector housing 328 also includes a plurality of connector contacts, such as connector contact 340. When the elongated device 300 is inserted into the port 330, the connector contacts 240 disposed in the connector housing 328 can be aligned with the terminals 310 of the elongated device 300 to electrically couple the lead extension 324 to the electrodes (134 of FIGS. 1 and 2) disposed along the lead (103 in FIGS. 1 and 2).

In at least some embodiments, the proximal end of the lead extension 324 is similarly configured and arranged as a proximal end of the lead 103 (or other elongated device 300). The lead extension 324 may include a plurality of electrically conductive wires (not shown) that electrically couple the connector contacts 340 to a proximal end 348 of the lead extension 324 that is opposite to the distal end 326. In at least some embodiments, the conductive wires disposed in the lead extension 324 can be electrically coupled to a plurality of terminals (not shown) disposed along the proximal end 348 of the lead extension 324. In at least some embodiments, the proximal end 348 of the lead extension 324 is configured and arranged for insertion into a connector disposed in another lead extension (or another intermediate device). In other embodiments (and as shown in FIG. 3B), the proximal end 348 of the lead extension 324 is configured and arranged for insertion into the control module connector 144.

Conventional electrical stimulation systems may be potentially unsafe for use with magnetic resonance imaging (“MRI”) due to the effects of electromagnetic fields in an MRI environment. A common mechanism for causing the electrical interactions between the electrical stimulation system and RF irradiation is common-mode coupling of an applied electromagnetic field that can act as a series of distributed sources along elongated conductive structures, such as leads, or conductors within a lead. Common-mode induced RF currents can reach amplitudes of greater than one ampere in MRI environments. Such currents can cause heating and potentially disruptive voltages within electronic circuits.

Some of the effects of RF irradiation may include, for example, inducing current in the lead, causing undesired heating of the lead that may potentially cause tissue damage, undesired or unexpected operation of electronic components, or premature failure of electronic components. Additionally, when an electrical stimulation system is used within an MRI scanner environment, the electrical interactions between the electrical stimulation system and the MRI may cause distortions in images formed by the MRI system.

In order to reduce the susceptibility of the lead to induced RF currents, one or more antenna properties (e.g., the ability to send or receive energy at certain frequencies or with certain field patterns), electromagnetic properties (e.g., inductance, capacitance, permittivity, or the like), or both, can be altered along the length of the lead.

Conductors connecting the terminals at one end of the lead to the electrodes (or other conductive contacts) at the other end of the lead can be arranged into one or more winding geometries along the length of the conductors to eliminate or reduce the effect of RF irradiation, such as applied electromagnetic fields generated during MRI. As herein described, the conductors extending along the length of the lead may include one or more coiled regions having winding geometries that alter antenna properties, electromagnetic properties, or both of the conductor or lead. The coiled regions can be disposed along the entire length of the conductors, or one or more portions thereof. Additionally, the winding geometry can be changed along the length of the lead in either a continuous or a discontinuous manner. It will be understood that any discussion below regarding conductors in a lead is also applicable, unless otherwise indicated, to conductors in a lead extension.

In some cases, adjusting the geometry of the coils may include changing a single geometry characteristic including, for example, changing one of the pitch, diameter, or number of filars. In other cases, adjusting the geometry of the coils may include changing different combinations of geometry characteristics including, for example, changing the pitch and the diameter, the pitch and the number of filars, the diameter and the number of filars, or the like.

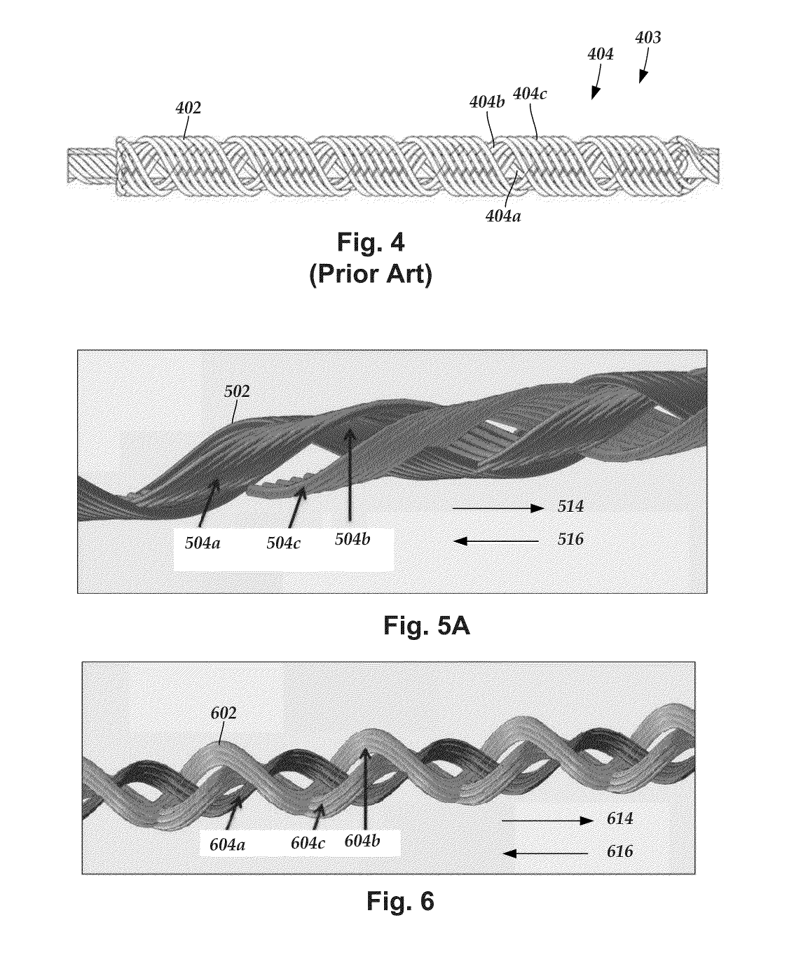

Turning now to FIG. 4-6, in at least some embodiments the conductors that extend between the electrodes and the terminals of a lead (or between the contacts and the terminals of a lead extension) have winding geometries that include one or more common-mode current suppression units (“units”) arranged in series. Examples of electrical stimulation systems with leads having conductors formed into such units are found in, for example, U.S. Patent Application Publication Nos. 2008/0243218; 2008/0262584; 2010/0076508; 2010/0094364; 2010/0256693; 2010/0326701; 2011/0009932; and 2011/0046700, all of which are incorporated by reference. Any number of units may be disposed along the longitudinal length of a conductor including, for example, one, two, three, four, five, six, seven, eight, nine, ten, twelve, fifteen, twenty, twenty-five, thirty, forty, fifty, or more units. It will be understood that any other numbers of units may be employed as well.

In at least some embodiments, each current suppression unit includes at least three connected conductor segments of the same conductor that are disposed within the same spatial region of the lead. Each unit includes a first conductor segment that extends in a first direction (e.g., forward) along a longitudinal length of an elongated member (e.g., a lead or lead extension) from a first position to a second position. Second, each unit includes a second conductor segment that extends in a second direction (e.g., reverse), opposite the first direction, from the second position back to a third position. Third, each unit includes a third conductor segment that extends in the first direction (e.g., forward) from the third position to a fourth position. In at least some embodiments, the second position is between the third and fourth positions. In at least some embodiments, the third position is between the first and second positions. This arrangement of conductor segments, with the middle segment extending in the opposite direction from the two end segments, can reduce or inhibit the induction of current from an external electromagnetic field, at least in part, because of the conductor segments extend in opposite directions.

The current suppression units may be electrically continuous such that the fourth position (e.g., endpoint) of a first unit is the first position (e.g., beginning point) of the next consecutive unit. At least one of the beginning points for the series of units may be a terminal or an electrode (or other conductive contact). Likewise, at least one of the endpoints for the series of units may be a terminal or an electrode (or other conductive contact). Typically, one or more of the conductor segments is coiled and, in at least some embodiments, all of the conductor segments are coiled. In at least some embodiments, the conductor segments are coiled around a liner, tube, mandrel or other structure. In at least some embodiments, the liner, tube, mandrel, or other structure defines a lumen that optionally is configured and arranged to receive a stiffening member (e.g., a stylet, or the like).

The conductors may have a single filament or be multi-filar, and they may be made of simple materials or composite constructions, such as drawn filled tubes. In preferred embodiments, the conductors are multi-filar drawn filled tubes. In some embodiments, two or more of the conductor segments can be individual pieces of conductive material that are electrically coupled (e.g., soldered or welded) together.

In some embodiments, the length of conductor used in the second conductor segment is at least 1.5, 1.75, 1.9, 2, 2.1, 2.25, or 2.5 times the length of either the first conductor segment or the third conductor segment. It will be recognized, however, that this ratio of conductor-segment lengths may vary among embodiments, particularly if the thickness of the conductor or thickness of conductor insulation disposed around the conductors is different for the different segments.

FIG. 4 schematically illustrates one embodiment of a prior art arrangement of multiple conductors 402 that are wound parallel to each other so that each conductor forms a current suppression unit. The conductors 402 include at least one region 403 that has at least one current suppression unit, such as unit 404, for each of the conductors. Each unit includes a first conductor segment 404a, a second conductor segment 404b, and a third conductor segment 404c that are stacked one on top of the other in a three layer arrangement. Conductor insulation is typically disposed over the conductors 402 to electrically isolate each of the conductors 402 from one another.

In contrast to the three layer arrangement of FIG. 4, the conductor segments can be wound together to form a structure that is primarily a single-layer structure with occasional two-layer regions where the second (e.g., reverse) conductor segment overlaps one or the other of the first or third (e.g., forward) conductor segments. Such arrangements can facilitate, for example, forming a lead with a smaller outer diameter than that using the arrangement illustrated in FIG. 4 due to the reduced number of layers of conductor segments.

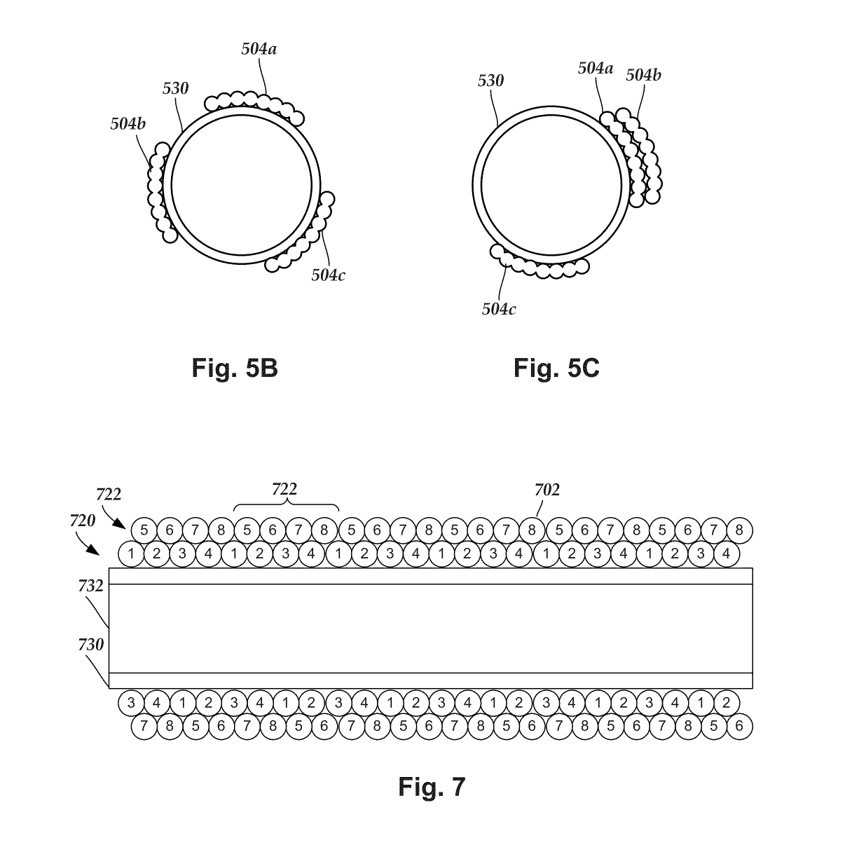

FIGS. 5A-5C illustrate one embodiment of such an arrangement using a ribbon of conductors with several conductors (e.g., eight conductors) disposed in a planar arrangement next to each other and coupled together, but conductively isolated, using a non-conductive material such as non-conductive plastic. FIG. 6 illustrates another embodiment of such an arrangement using a bundle of conductors where the conductors are bundled around a central element, such as one of the conductors or a central core of non-conductive material, or a central lumen. Whether in a ribbon, as illustrated in FIGS. 5A-5C, or a bundle, as illustrated in FIG. 6, the ribbon or bundle of conductors is wound to form current suppression units for the conductors.

In FIGS. 5A-5C and 6, each conductor 502, 602 includes a first conductor segment 504a, 604a that is coiled and extends in a first (e.g., forward) direction 514, 614, a second conductor segment 504b, 604b that is coiled and extends in a second (e.g., reverse) direction 516, 616 opposite the first direction, and a third conductor segment 504c, 604c that is coiled and extends in the first (e.g., forward) direction 514, 614. The first conductor segment 504a, 604a is attached to the second conductor segment 504b, 604b which, in turn, is attached to the third conductor segment 504c, 604c. In at least some embodiments, the conductor segments are coiled around central structure, such as a liner, tube, mandrel or other structure (see, structure 530 in FIGS. 5B and 5C).

In the arrangements of FIGS. 5A-5C and 6, the first conductor segment 504a, 604a and third conductor segment 504c, 604c are both wound in the same forward direction and at the same pitch so that the first and third conductor segments can be wound together in a single layer and at the same radial distance from a central longitudinal axis of the arrangement. For example, the first conductor segment 504a, 604a and third conductor segment 504c, 604c can be positioned 120 or 180 degrees (or any other suitable circumferential spacing) away from each other along the circumference defined by the desired radial distance. Because the first and third conductor segments are cowound at the same radial distance from the longitudinal axis, these conductor segments are disposed in the same layer.

The pitch of the first, second, and third conductor segments is selected so that there is space between the first and third conductor segments for the second conductor segment, along much of its length, to be disposed between the first and third conductor segments at the same radial distance from the central longitudinal axis (i.e., in the same layer as the first and third conductor segments), as illustrated in FIG. 5B. Because the second conductor segment is wound in the reverse direction, however, there will be regions where overlap occurs between the second conductor segment and either the first or third conductor segment. For example, if the pitch of the first, second, and third conductor segments is the same, this overlap may occur at four positions per revolution of the second conductor segment. The second conductor segment can then be positioned either above or beneath the first or third conductor segment that it overlaps, as illustrated in FIG. 5C. In these overlapping regions, the conductor segments will form two layers.

FIG. 7 illustrates another embodiment of a conductor arrangement to reduce or prevent induced current due to an external electromagnetic field, such as those produced during an MRI procedure. In the embodiment of FIG. 7, multiple conductors 702 are arranged so that one group 720 of conductors is wound around a central structure, such as a tube 730 (or a liner, mandrel, or other structure) and associated lumen 732, in one direction and a second group 722 of conductors, disposed on top of first group 720, is wound in the opposite direction. For example, the first group 720 can be wound clockwise and the second group 722 is then wound counterclockwise. Alternatively, the first group 720 can be wound counterclockwise and the second group 722 is then wound clockwise. It is thought that current that would be induced in one group of conductors would generate a field to oppose the current that would be induced in the other group of conductors (and vice versa) due to the opposite directions of winding. This would reduce or eliminate the induced current as compared to an arrangement in which all of the conductors were wound in the same direction.

The winding of the conductors is at a pitch that is greater than zero. At each position along the lead, the pitch for each of the conductors in a particular group is the same, although the pitch may vary along the length of the lead. In at least some embodiments, at each position along the lead, the pitch of both groups are the same.

In the illustrated embodiment, the first group 720 has four wires labeled ‘1’, ‘2’, ‘3’, and ‘4’, respectively and the second group 722 has four wires label ‘5’, ‘6’, ‘7’, and ‘8’, respectively. Other embodiments can include any number of wires in each group including, but not limited to, one, two, three, four, five, six, seven, eight, nine, ten, twelve, sixteen, or more wires. In addition, the number of wires in each group can be the same or can be different.

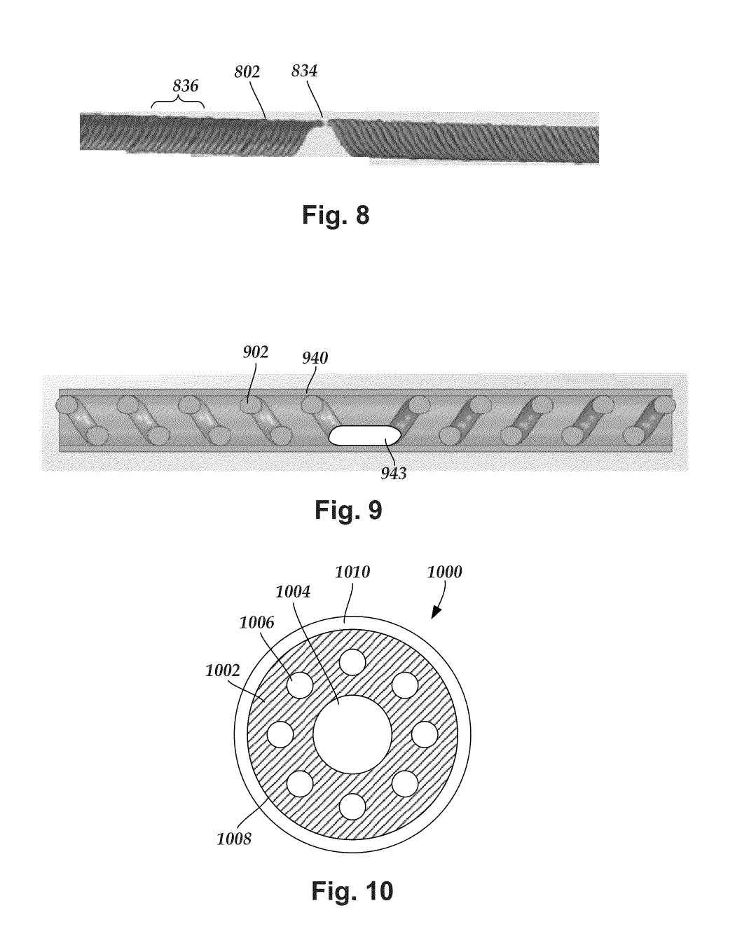

FIG. 8 illustrates another embodiment of a conductor arrangement to reduce or prevent induced current due to an external electromagnetic field, such as those produced during an MRI procedure. In the embodiment of FIG. 8, multiple conductors 802 (in the illustrated embodiment there are eight wires 836 in the form of a ribbon, such as that illustrated in FIG. 5A) are wound, optionally around a central structure (not shown), in a particular direction. Periodically, at transition segments 834, the direction of winding is reversed. For example, the group of wires is wound initially in a clockwise direction and then at the first transition segment the direction of winding is reversed to the counterclockwise direction. At the second transition segment, the direction of winding is again reversed, this time to the clockwise direction, and so forth.

The transition segments 834 can be relatively short (e.g., the width of the ribbon or no more than 1 mm, 2 mm, 5 mm, or more), which may be limited by the winding capability of the machinery used to form the coil of conductors, or transition segments 834 can be relatively long (e.g., 1 cm, 2 cm, or more). Any suitable distance between transition segments can be used including, for example, at least 1 cm, 2 cm, 5 cm, 10 cm, or more.

The conductors can be closely spaced as shown in FIG. 8 or the pitch of the conductors can be selected so that there is 1 mm or more spacing between adjacent coils of the ribbon. In addition, instead of using a ribbon of conductors, the conductors may be individually wound to form a similar arrangement or the conductors can be bundled similar to the conductors illustrated in FIG. 6. Any suitable number of conductors can be co-wound (as a ribbon or bundle or separately) including, but not limited to, two, three, four, five, six, seven, eight, nine, ten, twelve, sixteen, or more conductors.

FIG. 9 illustrates another embodiment of a conductor arrangement to reduce or prevent induced current due to an external electromagnetic field, such as those produced during an MRI procedure. In contrast to the embodiment of FIG. 8 in which multiple conductors are co-wound together, in the embodiment of FIG. 9 each individual conductor 902 is wound and then placed into a non-conductive jacket 940. The conductor 902 is wound in a first particular direction and then, periodically, at transition segments 934, the direction of winding is reversed. For example, the conductor is wound initially in a clockwise direction and then at the first transition segment the direction of winding is reversed to the counterclockwise direction. At the second transition section, the direction of winding is again reversed, this time to the clockwise direction, and so forth.

The transition segments 934 can be relatively short (e.g., the width of the ribbon or no more than 1 mm, 2 mm, 5 mm, or more), which may be limited by the winding capability of the machinery used to form the coil of conductors, or transition segments 934 can be relatively long (e.g., 1 cm, 2 cm, or more). Any suitable distance between transition segments can be used including, for example, at least 1 cm, 2 cm, 5 cm, 10 cm, or more.

Once each of the coiled conductors 902 is placed in its jacket 940, the coiled conductors 902 can be wrapped around a central structure (for example, tube 730 of FIG. 7 or a liner, mandrel, or other similar structure). This creates a coiled arrangement of conductors with each conductor arranged in a jacket and having windings with alternating directions (e.g., alternating between clockwise and counterclockwise).

In yet another embodiment, instead of placing the individual conductors shown in FIG. 9 into a jacket and then winding the conductors around a central structure, each individual conductor is wound as shown in FIG. 9 and then placed (absent the jacket) in a lumen of a multi-lumen structure. FIG. 10 illustrates such a multi-lumen structure as positioned within the body of a lead 1000. The lead 1000 includes an elongated multi-lumen conductor guide 1002. The multi-lumen conductor guide 1002 may extend an entire longitudinal length of the lead 1000 from the electrodes to the terminals. As shown in FIG. 10, the multi-lumen conductor guide 1002 defines a central lumen 1004 and a plurality of conductor lumens, such as conductor lumen 1006. The conductor lumens can have any suitable cross-sectional shape (e.g., round, oval, rectangular, triangular, or the like).

In at least some embodiments, the plurality of conductor lumens 1006 are encapsulated by the multi-lumen conductor guide 1002 such that the conductor lumens 1006 do not extend to an outer surface 1008 of the multi-lumen conductor guide 1002. Conductors (such as conductor 902 of FIG. 9) are individually disposed in the conductor lumens 1006 of the multi-lumen conductor guide 1002. The central lumen 1004 and the plurality of conductor lumens 1006 can be arranged in any suitable manner. In preferred embodiments, the conductor lumens 1006 are disposed in the multi-lumen conductor guide 1002 such that the conductor lumens 1006 are disposed around the central lumen 1004. In at least some embodiments, the lead 1000 may include a layer 1010 of covering material disposed over the outer surface 1008 of multi-lumen conductor guide 1002.

The central lumen 1004 may be configured and arranged to receive a stylet. The stylet can be used for assisting in insertion and positioning of the lead 1000 during implantation of the lead.

The conductor lumens 1006 are configured and arranged to receive conductors, which electrically couple the electrodes to the terminals. As indicated above the conductors will be wound as described with respect to the embodiment of FIG. 9. The conductor 902 is wound in a first particular direction and then, periodically, at transition segments 934, the direction of winding is reversed. For example, the conductor is wound initially in a clockwise direction and then at the first transition segment the direction of winding is reversed to the counterclockwise direction. At the second transition section, the direction of winding is again reversed, this time to the clockwise direction, and so forth.

The transition segments 934 can be relatively short (e.g., the width of the ribbon or no more than 1 mm, 2 mm, 5 mm, or more), which may be limited by the winding capability of the machinery used to form the coil of conductors, or transition segments 934 can be relatively long (e.g., 1 cm, 2 cm, or more). Any suitable distance between transition segments can be used including, for example, at least 1 cm, 2 cm, 5 cm, 10 cm, or more.

In at least some embodiments, each conductor lumen 1006 has a single conductor extending along the lumen. In other embodiments, two or more conductors can be disposed in a conductor lumen 1006. In such embodiments, the conductor lumens might have an oval or other non-circular shape in contrast to the conductor lumens illustrated in FIG. 10. Examples of such multi-lumen conductor guides, as well as other variations of suitable multi-lumen conductor guides, can be found at, for example, U.S. Patent Application Publications Nos. 2011/0230893 and 2012/0316615 and U.S. Provisional Patent Application Ser. Nos. 61/693,680 and 61/745,354, all of which are incorporated herein by reference. In at least some embodiments, the multi-lumen conductor guide 1002 defines more than one conductor lumen 1006, yet includes fewer conductor lumens 1006 than conductors 1020.

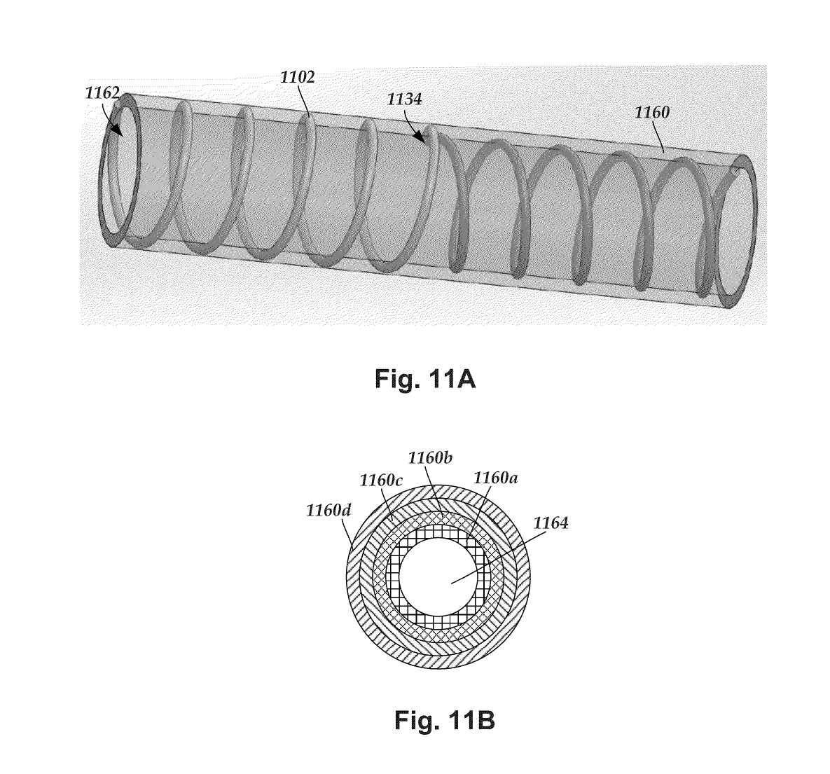

FIGS. 11A and 11B illustrate another embodiment of a conductor arrangement to reduce or prevent induced current due to an external electromagnetic field, such as those produced during an MRI procedure. As illustrated in FIG. 11A, a conductor 1102 is wound in a particular direction. Periodically, at transition segments 1134, the direction of winding is reversed. For example, the conductor is wound initially in a clockwise direction and then at the first transition segment the direction of winding is reversed to the counterclockwise direction. At the second transition segment, the direction of winding is again reversed, this time to the clockwise direction, and so forth.

The transition segments 1134 can be relatively short (e.g., the width of the ribbon or no more than 1 mm, 2 mm, 5 mm, or more), which may be limited by the winding capability of the machinery used to form the coil of conductors, or transition segments 1134 can be relatively long (e.g., 1 cm, 2 cm, or more). Any suitable distance between transition segments can be used including, for example, at least 1 cm, 2 cm, 5 cm, 10 cm, or more.

Once the conductor is wound it is embedded in an insulation material to form a tube 1160 with a central lumen 1162. The illustrated embodiment has one conductor per tube, but it will be understood that a tube may include multiple conductors (e.g., two, three, four, or more conductors). The multiple conductors may be formed in a ribbon shape (see, e.g., FIG. 5A) or in a bundle (see, e.g., FIG. 5B) or may be individual and disposed at different positions around the circumference of the tube (for example, 90, 120, or 180 degrees from each other).

The process is performed for each individual conductor, but the inner and outer diameters of the individual tubes 1160 are selected so that the final collections of tubes and wires can form a concentric arrangement. The concentric arrangement of tubes 1160a, 1160b, 1160c, 1160d is illustrated in FIG. 11B. This particular embodiment includes four tubes, but it will be understood that any number of tubes and any number of conductors may be used. For example, a lead may include two, three, four, five, six, seven, eight, nine, ten, twelve, or sixteen or more tubes disposed in a concentric arrangement.

The arrangement of FIG. 11B also includes a central lumen 1164 that can receive a stylet or allow delivery of a fluid, such as a pharmaceutical, along the lumen. It will be understood, however, that some embodiments may not include a central lumen and that the innermost tube may be a solid cylinder without a central lumen. In some embodiments, the outermost tube may form the outermost layer of the lead. In other embodiments, there may be one or more additional layers formed over the outermost tube.

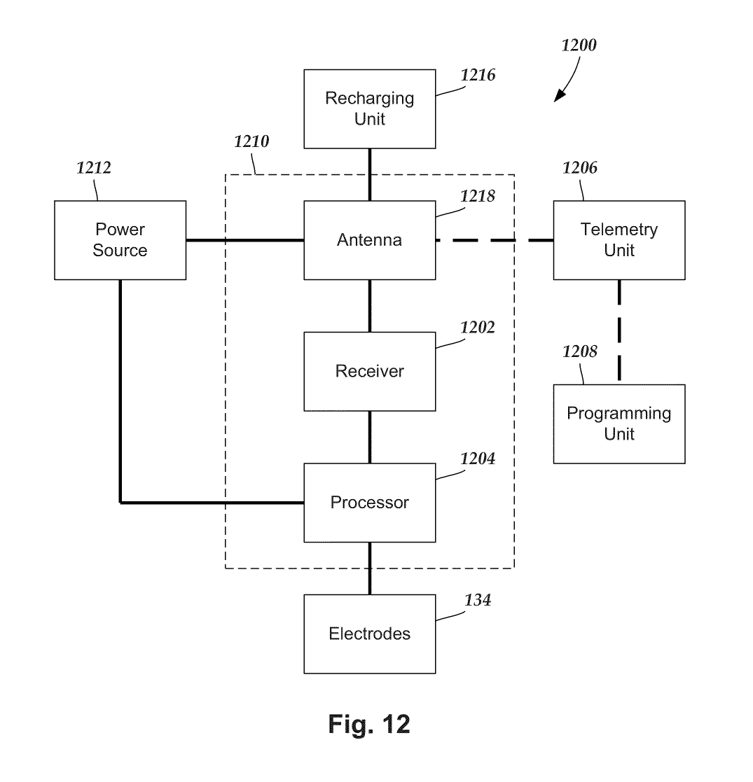

FIG. 12 is a schematic overview of one embodiment of components of an electrical stimulation system 1200 including an electronic subassembly 1210 disposed within a control module. It will be understood that the electrical stimulation system can include more, fewer, or different components and can have a variety of different configurations including those configurations disclosed in the stimulator references cited herein.

Some of the components (for example, a power source 1212, an antenna 1218, a receiver 1202, and a processor 1204) of the electrical stimulation system can be positioned on one or more circuit boards or similar carriers within a sealed housing of an implantable pulse generator, if desired. Any power source 1212 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1218 or a secondary antenna. The external power source can be in a device that is mounted on the skin of the user or in a unit that is provided near the user on a permanent or periodic basis.

If the power source 1212 is a rechargeable battery, the battery may be recharged using the optional antenna 1218, if desired. Power can be provided to the battery for recharging by inductively coupling the battery through the antenna to a recharging unit 1216 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. The processor 1204 is generally included to control the timing and electrical characteristics of the electrical stimulation system. For example, the processor 1204 can, if desired, control one or more of the timing, frequency, strength, duration, and waveform of the pulses. In addition, the processor 1204 can select which electrodes can be used to provide stimulation, if desired. In some embodiments, the processor 1204 selects which electrode(s) are cathodes and which electrode(s) are anodes. In some embodiments, the processor 1204 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1208 that, for example, allows modification of pulse characteristics. In the illustrated embodiment, the processor 1204 is coupled to a receiver 1202 which, in turn, is coupled to the optional antenna 1218. This allows the processor 1204 to receive instructions from an external source to, for example, direct the pulse characteristics and the selection of electrodes, if desired.

In one embodiment, the antenna 1218 is capable of receiving signals (e.g., RF signals) from an external telemetry unit 1206 which is programmed by the programming unit 1208. The programming unit 1208 can be external to, or part of, the telemetry unit 1206. The telemetry unit 1206 can be a device that is worn on the skin of the user or can be carried by the user and can have a form similar to a pager, cellular phone, or remote control, if desired. As another alternative, the telemetry unit 1206 may not be worn or carried by the user but may only be available at a home station or at a clinician's office. The programming unit 1208 can be any unit that can provide information to the telemetry unit 1206 for transmission to the electrical stimulation system 1200. The programming unit 1208 can be part of the telemetry unit 1206 or can provide signals or information to the telemetry unit 1206 via a wireless or wired connection. One example of a suitable programming unit is a computer operated by the user or clinician to send signals to the telemetry unit 1206.

The signals sent to the processor 1204 via the antenna 1218 and the receiver 1202 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1200 to cease operation, to start operation, to start charging the battery, or to stop charging the battery. In other embodiments, the stimulation system does not include the antenna 1218 or receiver 1202 and the processor 1204 operates as programmed.

Optionally, the electrical stimulation system 1200 may include a transmitter (not shown) coupled to the processor 1204 and the antenna 1218 for transmitting signals back to the telemetry unit 1206 or another unit capable of receiving the signals. For example, the electrical stimulation system 1200 may transmit signals indicating whether the electrical stimulation system 1200 is operating properly or not or indicating when the battery needs to be charged or the level of charge remaining in the battery. The processor 1204 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation lead, comprising:
at least one lead body having a distal end portion, a proximal end portion, and a longitudinal length;
a plurality of electrodes disposed along the distal end portion of the at least one lead body;
a plurality of terminals disposed along the proximal end portion of the at least one lead body; and
a plurality of conductors electrically coupling the plurality of terminals to the plurality of electrodes, wherein each of the conductors forms at least one current suppression unit, each current suppression unit comprises a first conductor segment wound in a coil and extending in a forward direction from a first point to a second point, a second conductor segment wound in a coil and extending in a reverse direction from the second point to a third point, and a third conductor segment wound in a coil and extending in the forward direction from the third point to a fourth point, wherein the first and third conductor segments are wound around a longitudinal axis with a first coil diameter and the second conductor segment is also wound around the longitudinal axis with the first coil diameter except at positions where the second conductor segment overlaps either the first conductor segment or the third conductor segment.

2. The electrical stimulation lead of claim 1, wherein the plurality of conductors are arranged in a ribbon with the conductors side-by-side.

3. The electrical stimulation lead of claim 1, wherein the plurality of conductors are arranged in a bundle with at least some of the conductors disposed in a ring around a central core.

4. The electrical stimulation lead of claim 1, wherein the plurality of conductors are arranged in a bundle with the plurality of conductors disposed in an arrangement with a central conductor and a remainder of the conductors disposed in a ring around the central conductor.

5. The electrical stimulation lead of claim 1, further comprising a central structure, wherein the plurality of conductors is wound around the central structure.

6. The electrical stimulation lead of claim 1, wherein the first conductor segment, second conductor segment, and third conductor segment are wound with a same pitch.

7. An electrical stimulating system comprising:
the electrical stimulation lead of claim 1;
a control module coupleable to the electrical stimulation lead, the control module comprising
a housing, and
an electronic subassembly disposed in the housing; and
a connector for receiving the electrical stimulation lead, the connector having a proximal end, a distal end, and a longitudinal length, the connector comprising
a connector housing defining a port at the distal end of the connector, the port configured and arranged for receiving the proximal end of the lead body of the electrical stimulation lead, and
a plurality of connector contacts disposed in the connector housing, the plurality of connector contacts configured and arranged to couple to at least one of the plurality of terminals disposed on the proximal end of the lead body of the electrical stimulation lead.

8. An electrical stimulation lead, comprising:
at least one lead body having a distal end portion, a proximal end portion, and a longitudinal length;
a plurality of electrodes disposed along the distal end portion of the at least one lead body;
a plurality of terminals disposed along the proximal end portion of the at least one lead body; and
a plurality of conductors electrically coupling the plurality of terminals to the plurality of electrodes, wherein each of the conductors forms at least one current suppression unit, each current suppression unit comprises a first conductor segment wound in a coil in a first winding direction selected from clockwise or counterclockwise, a transition segment connected to the first conductor segment, and a second conductor segment wound in a coil in a second winding direction that is opposite the first winding direction.

9. The electrical stimulation lead of claim 8, wherein the plurality of conductors are arranged in a ribbon with the conductors side-by-side.

10. The electrical stimulation lead of claim 8, further comprising a plurality of jackets, wherein each of the plurality of conductors is individually disposed in a corresponding one of the plurality of jackets.

11. The electrical stimulation lead of claim 10, further comprising a central structure extending along the lead within the lead body, wherein the conductors within the corresponding one of the jackets are wound around the central structure.

12. The electrical stimulation lead of claim 8, further comprising a multi-lumen conductor guide extending along the lead within the lead body, the multi-lumen conductor guide defining a plurality of conductor lumens extending along the multi-lumen conductor guide, wherein the plurality of conductors are disposed within the conductor lumens.

13. The electrical stimulation lead of claim 12, wherein only one of the plurality of conductors is disposed in each conductor lumen.

14. The electrical stimulation lead of claim 12, wherein the multi-lumen conductor guide defines a central lumen extending along the lead with the conductor lumens arrayed around the central lumen.

15. The electrical stimulation lead of claim 8, further comprising a plurality of non-conductive tubes arranged concentrically and extending along the lead, wherein each of the plurality of conductors is disposed in an individual one of the plurality of tubes.

16. An electrical stimulating system comprising:
the electrical stimulation lead of claim 8;
a control module coupleable to the electrical stimulation lead, the control module comprising
a housing, and
an electronic subassembly disposed in the housing; and
a connector for receiving the electrical stimulation lead, the connector having a proximal end, a distal end, and a longitudinal length, the connector comprising
a connector housing defining a port at the distal end of the connector, the port configured and arranged for receiving the proximal end of the lead body of the electrical stimulation lead, and
a plurality of connector contacts disposed in the connector housing, the plurality of connector contacts configured and arranged to couple to at least one of the plurality of terminals disposed on the proximal end of the lead body of the electrical stimulation lead.

17. An electrical stimulation lead, comprising:
at least one lead body having a distal end portion, a proximal end portion, and a longitudinal length;
a plurality of electrodes disposed along the distal end portion of the at least one lead body;
a plurality of terminals disposed along the proximal end portion of the at least one lead body; and
a plurality of conductors electrically coupling the plurality of terminals to the plurality of electrodes, wherein the plurality of conductors comprises a first group containing two or more of the conductors and a second group containing two or more of the conductors, wherein the first group of conductors are co-wound in a coil in a first winding direction to form a first layer and the second group of conductor are co-wound in a coil in a second winding direction to form a second layer disposed over the first layer, wherein the first winding direction is selected from clockwise or counterclockwise and the second winding direction is opposite the first winding direction.

18. The electrical stimulation lead of claim 17, wherein the first group of conductors and the second group of conductors contain a same number of conductors.

19. The electrical stimulation lead of claim 17, further comprising a central structure, wherein the first and second layers are disposed around the central structure.

20. An electrical stimulating system comprising:
the electrical stimulation lead of claim 17;
a control module coupleable to the electrical stimulation lead, the control module comprising
a housing, and
an electronic subassembly disposed in the housing; and
a connector for receiving the electrical stimulation lead, the connector having a proximal end, a distal end, and a longitudinal length, the connector comprising
a connector housing defining a port at the distal end of the connector, the port configured and arranged for receiving the proximal end of the lead body of the electrical stimulation lead, and
a plurality of connector contacts disposed in the connector housing, the plurality of connector contacts configured and arranged to couple to at least one of the plurality of terminals disposed on the proximal end of the lead body of the electrical stimulation lead.
